(19) United States
(12) Patent Application Publication
(10) Pub. No.: US 2003/0003110 A1
(43) Pub. Date: Jan. 2, 2003

(54) Immunomodulatory effective compositions, methods for the production thereof and their use

(76) Inventor: Angela Trenel, Laatzen (DE)

Correspondence Address: HORST M. KASPER, 13 FOREST DRIVE, WARREN, NJ, 07059, US

(21) Appl. No.: 10/218,644

(22) Filed: Aug. 14, 2002

Related U.S. Application Data
(63) Continuation-in-part of application No. PCT/DE01/00578, filed on Feb. 12, 2001.

(30) Foreign Application Priority Data
Feb. 14, 2000 (DE) 100 07 771.4

(51) Int. Cl. A61K 39/145; A61K 39/02; A61K 39/085; A61K 39/09; A61K 39/002; A61K 39/005; A61K 39/008

(52) U.S. Cl. 424/209.1; 424/243.1; 424/244.1; 424/264.1; 424/269.1; 424/234.1

(57) ABSTRACT

The invention relates to immunomodulatory effective microbiological compositions, to methods for the production thereof and to their use. The inventive microbiological immuno modulators are suited for use in active and passive immunization. The inventive compositions are primarily used as homeopathic medications in the areas of cardiac and circulatory disorders, hypertension or allergic ailments. The immunomodulatory effective microbiological compositions are comprised of equal parts of antigen and antitoxin suspensions and are potentiated using a potentiating solution.

### CROSS-REFERENCE TO RELATED APPLICATIONS

This application is a continuation-in-part application of another international application filed under the Patent Cooperation treaty Feb. 12, 2001, bearing Application No. PCT/DE01/00578, and listing the United States as a designated and/or elected country. The entire disclosure of this latter application, including the drawings thereof, is hereby incorporated in this application as if fully set forth herein.

### BACKGROUND OF THE INVENTION

1. Field of the Invention

The present invention relates to immunomodulatory effective microbiological compositions, methods for the production thereof and to their use. The microbiological immuno modulators according to the present invention are suited for use in active and passive immunization. As homeopathic medication, the main applications of the microbiological immunomodulators are situated in the fields of heart disease and circulatory disorders, hypertension and allergic maladies.

2. Brief Description of the Background of the Invention Including Prior Art

Immunomodulatory effective compositions have been described several times in the patented literature, for example in the printed Patent Document WO 98/56405 as a pharmaceutical composition. Modulators of the human immune system are subject matter of the European patent application EP 0109089, wherein the modulators have been isolated from dialyzates of leucocyte extracts. Methods and compositions for modulating the immune answer are mentioned in the printed Patent document WO 94/226114. Compositions for treatment of immune weakness can be gathered from the international patent application WO 94/010980, wherein the compositions for example comprise an anti-cytokinesis antibody. The printed Patent document WO 95/07933 describes compositions which contain antibodies or antibody like molecules of primary antigens. A possible immuno active composition is reported in the printed Patent document WO 98/52605, wherein the possible immuno active composition comprises an antigen or an antigen inducing substance as well as a carrier and is to effect an increase in the immune response. The patent application WO 98/57620 describes a composition for modulating the immune response in mammals. A stereo isomer of inositol, its derivatives, salts and mixtures is recited there as a therapeutic agent. This agent increases or, respectively, suppresses the B-and/or T- lymphocyte activity. Antigen inducers and anti-toxins having high selectivity and specificity and capable of being employed as regulators of the immune response are subject matter of the printed Patent document WO 98/32431.

### SUMMARY OF THE INVENTION

1. Purposes of the Invention

It is an object of the present invention to develop new pharmaceutical compositions and methods for their production.

These and other objects and advantages of the present invention will become evident from the description which follows.

2. Brief Description of the Invention

The present invention provides and the object of the invention is accomplished by the furnishing of immunomodulatory effective compositions. The immunomodulatory compositions are characterized in that they are composed out of equal parts of antigen suspensions and anti-toxin suspensions and are potentiated with a potentiating solution.

Here the antigens are obtained from the following microorganisms:

from mycobacterium tuberculosis, mycobacterium tuberculosis typus bovis or

luctococcus lactis

virus influenza

streptococcus pyogenes, -oralis, -pneumoniae

staphylococcus aureus sub. aureus, -epidermis

treponema pallidum

plusmodium malaria

haemophilus influenza or

klebsiella pneumoniae

and the anti-toxin suspensions by immunization of mammals—such as rats, mice, horses, in particular rabbits—with the corresponding antigen suspensions.

The compositions according to the present invention contain a potentiating solution out of ethanol and/or thymol and cleaned water. In addition the following materials can be contained in the potentiating solution: iodine, hydrochloric acid HCl, and/or sugar syrup. Advantageously equal parts of the antigen suspension and the antitoxin suspension are potentiated to D9 (dil. 9).

The invention compositions represent microbiological immunomodulators. They contain antigens and anti-toxins of different bacteria strains. The antitoxin suspensions contain a phenol-lactic acid mixture.

The spectrum of indication of these compositions is extraordinarily broad. A large therapeutic breadth results here caused by the fact that many diseases are based on mixed infections, interferences of the immune system, allergies or on autoimmune diseases.

The immune therapy with these compositions opens up the field of diseases with so-called “unknown origins”. Frequently tuberculo toxicoses or luetic-toxic hereditary weakness are hiding behind “unknown origins”.

The compositions according to the present invention can be applied percutaneously, that is the compositions are rubbed in forcefully by the patient with himself or herself at a skin location as tender as possible with the thumb balls. This can be the elbows, the inner sides of the thighs or the abdominal skin. In case of infants, the droplets can be rubbed with the forearm of the child into the abdominal skin.

The invention method for producing the compositions comprises the following steps:

a) production of antigen-suspensions from killed cultures of microorganisms

b) production of antitoxin suspensions by immunization of animals with the corresponding antigen suspensions

c) mixing of antigen suspensions and the corresponding antitoxin suspensions in the ratio 1:1 and furnishing with the potentiating solution.

The potentiating solution comprises ethanol and/or thymol and clean water. Clean water is defined as aqua purificata for the production of medicines in DAB 10, Base delivery 1991. The clean water aqua purificata is produced from drinking water by distillation, by employing of ion exchangers, or by another suitable method. The potentiating solution can additionally contain sugar syrup and/or ethanolic iodine tincture and/or hydrochloric acid HCl.

The features of the invention can be gathered in addition to the claims also from the description, wherein the individual features in each case by themselves or as several in the form of combinations represent advantageous protectable embodiments, for which embodiments protection is applied for with this document. The combination comprises known elements (potentiation, obtaining from microorganisms strains) and the new elements (compositions, which are composed out of equal parts of antigen suspension and antitoxin suspension and contained a potentiating solution; phenol-lactic acid mixtures), which influence themselves mutually and result in their new overall effect in an advantage (synergistic effect) and the desired success, wherein the desired success comprises that now microbiological immunomodulators are available for active immunization and for passive immunization.

A further combination feature comprises that the solutions number 1 through 20 according to the present invention can be applied together by combination amongst themselves.

The invention is to be explained in more detail by way of embodiments examples without being limited to these embodiment examples.

### EMBODIMENT EXAMPLES

### Example 1

Production of the Antigen Suspension

An above recited strain of bacteria is cultivated in a suitable nutrient medium to up to 10⁷ germs per gram. This culture is autoclaved with five milliliters 1-n-sodium hydroxide solution (DAB—German medication book—Deutsches Arzneibuch) for 15 minutes at a temperature of 121 degrees centigrade and one atmosphere pressure after a germ bacterial determination, (prescription V.2.6.12 EuAB—European medication book) and in the following set to a pH 7 with 1-n-hydrochloric acid HCl. The autoclaved culture is tested with respect to sterility according to EuAB (prescription 2.6.1). This suspension is the antigen suspension and serves as an original tincture for potentiating.

### Example 2

Production of the Antitoxin Suspension

A: three rabbits with the weight of in each case more than 2.5 kg are employed for the production of this original tincture for the immunization. Each rabbit is investigated by a veterinarian after a two-week quarantine and is released for the immunization only after issuance of a health certificate.

B: 2 milliliters of the “antigen suspension” are subcutaneously treated for each rabbit (boosting after 7 and possibly further days). The immunization is finished as soon as specific antibodies are detectable in a bacteria agglutination test.

C: 24 milliliters blood are removed lege artis from the rabbit with the highest antibody titer, of which 24 milliliters there are 22 milliliters mixed immediately after the removal in 88.0 milliliters phenol-lactic acid mixture (0.5 percent liquid phenol DAC (German medicine codecs—Deutscher Arzneimittel Kodex), 0.5 percent lactic acid DAB 10 (G/V). This mixture (110, 0 milliliters) is slightly shaken for a time period of two minutes.

D: an additional 990.0 milliliters of the phenol-lactic acid mixture described in C are added to the 110.0 milliliters of blood—phenol-lactic acid mixture according to C. This mixture (1100.0 milliliters designated as “stock solution”) is slightly shaken for two minutes.

E: 55.0 milliliters of the stock solution according to D are thinned down with 1045.0 milliliters of the phenol-lactic acid mixture described in C and are autoclaved for 15 minutes at 121 degrees centigrade and one atmosphere pressure. The mixture (totaling 1100.0 milliliters) is tested for sterility according to EuAB (prescription 2.6.1).

F: 1000.0 milliliters of the mixture according to E are furnished with 5.0 milliliters nitric acid 65 percent DAB 10 and maintained boiling for a time period of 30 minutes. The still hot mixture is neutralized initially with sodium hydroxide DAB 10 and then with sodium carbonate DAB 10, set to a pH 8.0 and is furnished with 10.0 milliliters ethanol 96 percent DAB 10 and 50.0 milliliters glycerol 85 percent DAB 10. In the following the solution is built to 1000.0 milliliters with water for injection purposes DAB 10. This solution is the antitoxin suspension, which serves as original tincture for potentiating.

### Example 3

Potentiating

The antigen suspension and the antitoxin suspension are mixed in equal parts (m/m) and are potentiated with the potentiating solution according to the prescriptions of the HAB1 (homeopathic medication book—Homeopathic Arzneibuch).

### Example 4

Solutions

Solution 1

Compositions: 1 ml contains antigens dil. D9 out of mycobacterium tuberculosis typus bovis. “dil. D9” has the meaning dil. for latin dilitus—dilute, and D9 refers to HAB stepwise potentiating, for example D1=1:10, D2=1:100 etc. . . . D9=1:1,000,000,000.

Field of application: hypertension, heart disease, premature geriatric complaints with gland disorders and metabolic disorders, arteriosclerosis, neuropathy, paradontosis, prostate diseases.

Solution 2

Composition: 1 ml contains antigens dil. D9 and antitoxin dil. D9 from virus influenza, haemophilus influencae, klebsiella pneumoniae.

Fields of application: common cold, influenza, angina, furunculosis, inflammations.

Solution 3

Composition: 1 ml contains antigens dil. D9 and antitoxins dil. D9 out of staphylococcus aureus sub. aureus, streptococcus pneumoniae.

Fields of application: circulatory disorders, venous diseases, colics, allergic sufferings such as asthma, hay fever and the like.

Solution 4

Composition: 1 ml contains antigens dil. D9 from mycobacterium tuberculosis typus bovis, luctococcus lactis, streptococcus pyogenes, -oralis, -pneumoniae, staphylococcus aureus sub. aureus, -epidermis.

Fields of application: circulatory disorders, venous diseases, allergic sufferings in combination with solution 1 and solution 3, pain treatment.

Solution 5

Composition: 1 ml contained antigens dil. D9 and antitoxins dil. D9 out of mycobacterium tuberculosis typus bovis, streptococcus pyogenes.

Fields of application: rheumatism, gout, iscialgia, neuralgia.

Solution 6

Composition: 1 ml contains antigens dil D9 and antitoxins dil. D9 out of mycobacterium tuberculosis, mycobacterium tuberculosis bovis, streptococcus pneumoniae.

Fields of application: scrofulosis and tuberculosis and their late and larval (masked) forms of expression such as asthma, eczema, rheumatism, migrain, and the like. The common application together with solution 5 is advantageous in case of rheumatic forms of disease. Solution 6 is the agent of choice in case of skin diseases and inflammation of lymph nodes during childhood based on allergy (scrofulosis).

Solution 7

Composition:

1 ml contains antigens dil. D9 and antitoxins dil. D9 from treponima pallidum.

Solution 8

Composition:

1 ml contains antigens dil. D9 and antitoxins dil. D9 from plusmodium malaria.

Solution 9

Composition: 1 ml contains antigens dil. D9 from mycobacterium tuberculosis typus bovis, lactococcus lactis, streptococcus pyogenes, -oralis, -pneumoniae, staphylococcus aureus sub. aureus, -epidermis.

Fields of application: testing of all focal infections at teeth, tonsils, nasal sinuses and the like.

Solution 10

Composition: 1 ml contains antigens dil. D9 out of staphylococcus aureus sub. aureus, streptococcus pneumoniae.

### Example 5

Production of the Antigen Suspension

An above recited strain of bacteria is cultivated in a suitable nutrient medium to from about 10⁶ up to 10⁷ germs per gram. This culture is autoclaved with from about two to ten milliliters 1-n-sodium hydroxide solution (DAB—German medication book—Deutsches Arzneibuch) for from about 5 to 60 minutes at a temperature of from about 110 to 130 degrees centigrade and from about 0.5 to two atmospheres pressure after a germ bacterial determination, (prescription V.2.6.12 EuAB—European medication book) and in the following set to a pH from about 6 to 8 with preferably 1-n-hydrochloric acid HCl. The autoclaved culture is tested with respect to sterility according to EuAB (prescription 2.6.1). This suspension is the antigen suspension and serves as an original tincture for potentiating.

### Example 6

Production of the Antitoxin Suspension

A: three rabbits with the weight of in each case more than 2.5 kg are employed for the production of this original tincture for the immunization. Each rabbit is investigated by a veterinarian after a two-week quarantine and is released for the immunization only after issuance of a health certificate.

B: From about 1 to 5 milliliters of the “antigen suspension” are subcutaneously treated for each rabbit (boosting after 7 and possibly further days). The immunization is finished as soon as specific antibodies are detectable in a bacteria agglutination test.

C: from about 10 to 100 milliliters blood are removed lege artis from the rabbit with the highest antibody titer, of which 10 to 100 milliliters there are 9 to, respectively, 90 milliliters mixed immediately after the removal in from about 40 to, respectively 400.0 milliliters phenol-lactic acid mixture (from about 0.1 to 1.0 percent liquid phenol DAC (German medicine codecs—Deutscher Arzneimittel Kodex), from about 0.1 to 1.0 percent lactic acid DAB 10 (G/V). This mixture (from about 44 to, respectively 440.0 milliliters) is slightly shaken for a time period of two minutes.

D: an additional from about 39.6 to, respectively, 396.0 milliliters of the phenol-lactic acid mixture described in C are added to the from about 44.0 to, respectively 440.0 milliliters of blood—phenol-lactic acid mixture according to C. This mixture (from about 44.0 to, respectively, 440.0 milliliters designated as “stock solution”) is slightly shaken for two minutes.

E: from about 22.0 to, respectively, 220.0 milliliters of the stock solution according to D are thinned down with from about 209.0 to 2090.0, respectively, milliliters of the phenol-lactic acid mixture described in C and are autoclaved for a time period of from about 5 to 60 minutes at from about 110 to 130 degrees centigrade and from about 0.5 to 2.0 atmosphere pressure. The mixture (totaling from about 440.0 to, respectively 4400.0 milliliters) is tested for sterility according to EuAB (prescription 2.6.1).

F: from about 400.0 to 4000.0 milliliters of the mixture according to E are furnished with from about 2.0 to, respectively 10.0 milliliters nitric acid from about 50 to 75 percent DAB 10 and maintained boiling for a time period of from about 10 to 120 minutes. The still hot mixture is neutralized initially with sodium hydroxide DAB 10 and then with sodium carbonate DAB 10, set to a pH from about 7.0 to 9.0 and is furnished with from about 4.0 to 40.0 milliliters ethanol for example 96 percent DAB 10 and from about 20.0 to 100.0 milliliters glycerol for example from about 65 to 95 percent DAB 10. In the following the solution is built to from about 400.0 to 4000.0 milliliters with water for injection purposes DAB 10. This solution is the antitoxin suspension, which serves as original tincture for potentiating.

### Example 7

Potentiating

The antigen suspension and the antitoxin suspension are mixed in equal parts (m/m) and are potentiated with the potentiating solution according to the prescriptions of the HAB1 (homeopathic medication book—Homeopathic Arzneibuch).

### Example 8

Solutions

Solution 11

Compositions: 1 ml contains antigens dil. D9 out of mycobacterium tuberculosis typus bovis.

Field of application: hypertension, heart disease, premature geriatric complaints with gland disorders and metabolic disorders, arteriosclerosis, neuropathy, paradontosis, prostate diseases.

Solution 12

Composition: 1 ml contains antigens dil. D9 and antitoxin dil. D9 from virus influenza, haemophilus influencae, klebsiella pneumoniae.

Fields of application: common cold, influenza, angina, furunculosis, inflammations.

Solution 13

Composition: 1 ml contains antigens dil. D9 and antitoxins dil. D9 out of staphylococcus aureus sub. aureus, streptococcus pneumoniae.

Fields of application: circulatory disorders, venous diseases, colics, allergic sufferings such as asthma, hay fever and the like.

Solution 14

Composition: 1 ml contains antigens dil. D9 from mycobacterium tuberculosis typus bovis, luctococcus lactis, streptococcus pyogenes, -oralis, -pneumoniae, staphylococcus aureus sub. aureus, -epidermis.

Fields of application: circulatory disorders, venous diseases, allergic sufferings in combination with solution 11 and solution 13, pain treatment.

Solution 15

Composition: 1 ml contained antigens dil. D9 and antitoxins dil. D9 out of mycobacterium tuberculosis typus bovis, streptococcus pyogenes.

Fields of application: rheumatism, gout, iscialgia, neuralgia.

Solution 16

Composition: 1 ml contains antigens dil D9 and antitoxins dil. D9 out of mycobacterium tuberculosis, mycobacterium tuberculosis bovis, streptococcus pneumoniae.

Fields of application: scrofulosis and tuberculosis and their late and larval (masked) forms of expression such as asthma, eczema, rheumatism, migrain, and the like. The common application together with solution 15 is advantageous in case of rheumatic forms of disease. Solution 16 is the agent of choice in case of skin diseases and inflammation of lymph nodes during childhood based on allergy (scrofulosis).

Solution 17

Composition:

1 ml contains antigens dil. D9 and antitoxins dil. D9 from treponima pallidum.

Solution 18

Composition:

1 ml contains antigens dil. D9 and antitoxins dil. D9 from plusmodium malaria.

Solution 19

Composition: 1 ml contains antigens dil. D9 from mycobacterium tuberculosis typus bovis, lactococcus lactis, streptococcus pyogenes, -oralis, -pneumoniae, staphylococcus aureus sub. aureus, -epidermis.

Fields of application: testing of all focal infections at teeth, tonsils, nasal sinuses and the like.

Solution 20

Composition: 1 ml contains antigens dil. D9 out of staphylococcus aureus sub. aureus, streptococcus pneumoniae.

Fields of application: testing of all focal infections at teeth, tonsils, nasal sinuses and the like.

Literature Suggestion

The data in the recited medicine books

EuAB—Europaeisches Arzneibuch

DAB—Deutsches Arzneibuch

DAC—Deutscher Arzneimittel Codex

HAB—Homoeopathisches Arzneibuch

refer to the in each case valid version.

It will be understood that each of the elements described above, or two or more together, may also find a useful application in other types of immunomodulatory compositions and treatment procedures differing from the types described above.

While the invention has been illustrated and described as embodied in the context of a immunomodulatory effective composition, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. An immunomodulatory effective composition comprising antigen suspensions and antitoxin suspensions in equal parts, wherein the antigen suspensions and antitoxin suspensions in equal parts are potentiated with a potentiating solution.

2. The composition according to claim 1 wherein antigen suspensions are obtained from a microorganism of the group consisting of:
from mycobacterium tuberculosis, mycobacterium tuberculosis typus bovis,
luctococcus lactis,
virus influenza,
streptococcus pyogenes, -oralis, -pneumoniae,
staphylococcus aureus sub. aureus, -epidermis,
treponema pallidum,
plusmodium malaria,
haemophilus influenza,
klebsiella pneumoniae and mixtures thereof.

3. The composition according to claim 1 wherein the antitoxin suspensions are obtained by immunizing of mammals with the corresponding antigen suspensions.

4. Compositions according to claim 1, wherein the antitoxin suspensions contain a phenol-lactic acid mixture.

5. The composition according to claim 4, wherein the phenol-lactic acid mixture contains from about 0.1 to 1.0 percent phenol and from about 0.1 to 1.0 percent lactic acid.

6. The composition according to claim 5 wherein the potentiating solution contains ethanol and/or thymol and purified water.

7. The composition according to claim 6 wherein the potentiating solution contains additionally a member selected from the group consisting of sugar syrup, ethanolic iodine tincture, hydrochloric acid HCl, and mixtures thereof.

8. The composition according to claim 1 wherein a potentiation occurs.

9. A method for the production of compositions according to claim 1 through 8, comprising the following steps:
producing antigen suspensions out of killed cultures of a microorganism of the group consisting of:
mycobacterium tuberculosis, mycobacterium tuberculosis typus bovis,
luctococcus lactis,
virus influenza,
streptococcus pyogenes, -oralis, -pneumoniae,
staphylococcus aureus sub. aureus, -epidermis,
treponema pallidum,
plusmodium malaria,
haemophilus influenza,
klebsiella pneumoniae and mixtures thereof;
b) producing antitoxin suspensions by immunizing of animals with the corresponding antigen suspensions;
c) mixing of antigen suspensions and the corresponding antitoxin suspensions in a ratio 1:1 and furnishing with the potentiating solution containing a member selected from the group consisting of ethanol, thymol, purified water, and mixtures thereof.

10. The method according to claim 9 wherein
a) microorganisms strains are cultivated from 10⁶ up to 10⁷ germs per gram for the production of antigen suspensions and these cultures are set to a pH from about 6 to 8;
b) the corresponding antigen suspensions are entered subcutaneously to animals for the production of the antitoxin suspensions.

11. The method according to claim 10 wherein blood, which was removed from the animals, is received in a phenol-lactic acid mixture for the production of the antitoxin suspensions.

12. The method according to claim 9 wherein the suspensions are obtained from a member selected from the group consisting of lactic acid, glycerol, ethanol, water for injection purposes, purified water, sodium hydroxide, sodium carbonate, hydrochloric acid HCl, phenol, nitric acid, and mixtures thereof.

13. The method according to claim 12 further comprising the steps:
autoclaving blood of immunized animals in a phenol-lactic acid mixture,
heating the autoclaved blood together with nitric acid,
neutralizing the blood with the nitric acid;
setting the neutralized liquid to a pH from 7.0 to 9.0;
furnishing the liquid with ethanol and glycerol; and
filling the liquid up with water for injection purposes for the production of the antitoxin suspensions.

14. The method according to claim 13 wherein the antigen suspension and the antitoxin suspension are mixed in equal parts and are potentiated.

15. The method according to claim 13 further comprising
potentiating the liquid with ethanol.

16. The method according to claim 13 further comprising
potentiating the liquid without ethanol and with a member selected from the group consisting of thymol, sirupus simplex, tincture iodine, hydrochloric acid, purified water, and mixtures thereof.

17. The method according to claim 13 further comprising
treating circulatory disorders, cardiac disease, hypertension, inflammations, common colds, arteriosclerosis, allergic ailments as well as during testing of focal infections at teeth, tonsils or nasal sinuses.

18. The method according to claim 13 further comprising
treating venous diseases, premature geriatric complains in connection with gland disorders and metabolic disorders, neuralgias, paradontosis, prostate disease, influenza, angina, eczema, furunculosis, colics, asthma, hay fever, rheumatism, gout, iscialgia, neuralgia, migrain, scrofulosis or tuberculosis.
